(19) United States
(12) Patent Application Publication
(10) Pub. No.: US 2013/0177956 A1
(43) Pub. Date: Jul. 11, 2013

(54) MICROORGANISMS FOR 1,3-PROPANEDIOL PRODUCTION USING HIGH GLYCERINE CONCENTRATION

(71) Applicant: Rainer Figge, Le Crest (FR)

(72) Inventor: Rainer Figge, Le Crest (FR)

(73) Assignee: METABOLIC EXPLORER, Saint Breauzire (FR)

(21) Appl. No.: 13/823,709

(22) PCT Filed: Nov. 10, 2011
(86) PCT No.: PCT/EP2011/069789

§ 371 Date: Mar. 14, 2013

Related U.S. Application Data
(60) Provisional application No. 61/412,162, filed on Nov. 10, 2010.

(30) Foreign Application Priority Data
Nov. 10, 2010 (EP) 10306234.5

(51) Int. Cl. C12N 15/74

(52) U.S. Cl. CPC C12N 15/74
USPC 435/158; 435/252.3

(57) ABSTRACT

The present invention is related to a population of Clostridium acetobutylicum useful for the production of 1,3-propanediol (PDO), wherein said population comprises at least one strain of a Clostridium acetobutylicum sp. comprising mutations selected among the mutations identified in table 1, wherein relative percentages of said mutations are selected among specific genes.

The present invention concerns a new modified microorganism for the production of 1,3-propanediol. This microorganism is adapted for growth and production of 1,3-propanediol from a culture medium with high glycerine content and specifically with a high concentration of industrial glycerine. The invention also concerns culture conditions of said adapted microorganisms and process for the production of 1,3-propanediol. The invention concerns, finally, 1,3-propanediol produced by the modified microorganism and its applications.

### BACKGROUND OF THE INVENTION

1,3-propanediol (PDO), also called trimethylene glycol or propylene glycol, is one of the oldest know fermentation products. It was originally identified as early as 1881 by August Freund in a glycerine fermented culture containing Clostridium pasteurianum. PDO is a typical product of glycerine fermentation and has been found in anaerobic conversions of other organic substrates. Only very few organisms, all of them bacteria, are able to form it. They include enterobacteria of the genera Klebsiella (K. pneumoniae), Enterobacter (E. agglomerans) and Citrobacter (C. freunddi), Lactobacilli (L. brevis and L. buchneri) and Clostridia of the C. butyricum and the C. pasteurianum group.

PDO, as a bifunctional organic compound, may potentially be used for many synthesis reactions, in particular as a monomer for polycondensations to produce polyesters, polyethers, polyurethanes, and in particular, polytrimethylene terephtalate (PTT). These structure and reactivity features lead to several applications in cosmetics, textiles (clothing fibers or flooring) or plastics (car industry and packing or coating).

PDO can be produced by different chemical routes but they generate waste stream containing extremely polluting substances and the cost of production is high. Thus, chemically produced PDO can not compete with the petrochemically available diols like 1,2-ethanediol, 1,2-propanediol and 1,4-butanediol. To increase this competitiveness, in 1995, DuPont started a research program for the biological conversion of glucose to PDO. Although this process is environmentally friendly it has the disadvantage to i) use vitamin B12 a very expensive cofactor and ii) be a discontinuous process due to the instability of the producing strain.

Due to the availability of large amounts of glycerine produced by the bio-diesel industry, a continuous, vitamin-B12-free process with a higher carbon yield would on the contrary, be advantageous.

It is known in the art that PDO can be produced from glycerine, an unwanted by-product of the biodiesel production that contains roughly 80-85% of glycerine mixed with salts and water.

C. butyricum was previously described as being able to grow and produce PDO from industrial glycerine in batch and two-stage continuous fermentation (Papanikolaou et al., 2000). However, at the highest glycerine concentration, the maximal PDO titer obtained was 48.1 g·L⁻¹ at a dilution rate of 0.02 h⁻¹, meaning a productivity of 0.9 g·L⁻¹·h⁻¹. The cultures were conducted with a maximum glycerine concentration in the fed medium of 90 g·L⁻¹ and in the presence of yeast extract, a costly compound containing organic nitrogen that is known by the man skilled in the art to help increase bacterial biomass production.

Application WO2006/128381 discloses the use of this glycerine for the production of PDO with batch and fed-batch cultures using natural PDO producing organisms such as Klebsiella pneumoniae, C. butyricum or C. pasteuricum. Furthermore, the medium used in WO2006/128381 also contains yeast extract. As described in this patent application, the maximal productivity reached was comprised between 0.8 and 1.1 g.

The performance of a C. acetobutylicum strain modified to contain the vitamin B12-independent glycerine-dehydratase and the PDO-dehydrogenase from C. butyricum, called “C. acetobutylicum DG1 pSPD5” has been described in Gonzalez-Pajuelo et al., 2005. This strain originally grows and produces PDO in a fed medium containing up to 120 g·l⁻¹ of pure glycerine. In addition, analyses in a fed medium containing a maximum of 60 g·l⁻¹ of pure or industrial glycerine did not point out to any differences. These results have been obtained in presence of yeast extract. Moreover, no test was performed with concentrations of industrial glycerine higher than 60 g·l⁻¹. When comparing a wild-type C. butyricum to the modified microorganism “C. acetobutylicum DG1 pSPD5”, a globally similar behaviour was observed.

In patent application PCT/EP2010/056078 the inventors have described a process to adapt the strain of C. acetobutylicum DG1 pSPD5 such as described in Gonzalez-Pajuelo et al. (2005) to grow in a medium with a high concentration of industrial glycerine and without yeast extract. The resulting strain is able to produce PDO in medium containing up to 120 g·l⁻¹ of industrial glycerine with a titer up to 53.5 g·L⁻¹ of PDO, a yield up to 0.53 g·g⁻¹ and a productivity up to 2.86 g·L⁻¹·h⁻¹.

In the present patent application, the inventors highlight the main genetics modifications of the adapted microorganism useful for the production of PDO, such as obtained after adaptation in presence of high concentration of industrial glycerine.

### BRIEF DESCRIPTION OF THE INVENTION

The present invention concerns a population of Clostridium acetobutylicum useful for the production of 1,3-propanediol (PDO), wherein said population comprises at least one strain of a Clostridium acetobutylicum sp. comprising mutations selected among the mutations identified in Table 1, wherein relative percentages of said mutations are selected among the following gene families:

| Gene family and function | Minimum % |
|---|---|
| Transcription translation regulation | 12-15 |
| Transporters | 10-12 |
| Hypothetical proteins | 8-11 |
| Energy metabolism | 7-10 |
| Intergenic | 7-10 |
| Carbohydrate metabolism | 5-7 |
| Membrane proteins | 2-5 |
| Nucleic acid metabolism | 2-5 |
| Amino acid metabolism | 1-3 |
| Cell division | 1-3 |
| Sporulation | 1-3 |
| Cell adhesion | 0-1 |
| Cellulase | 0-1 |
| Glycerol metabolism | 0-1 |
| Lipid metabolism | 0-1 |
| Proteases/Peptidases | 0-1 |
| Cell motility | 0-1 |

Particularly, the population of the invention comprises at least one strain of Clostridium acetobutylicum selected among the group consisting of:
strain DG1 pSPD5 PD0001VE05c01 deposited at CNCM under accession number I-4378;
strain DG1 pSPD5 PD0001VE05c05 deposited at CNCM under accession number I-4379;
strain DG1 pSPD5 PD0001VE05c07 deposited at CNCM under accession number I-4380.
   CNCM means “Collection Nationale de Cultures de Microorganismes” at the Pasteur Institute, Paris.

In a particular embodiment of the invention, the population comprises the above strains further mutated with at least one of the following point mutations:
C is replaced with T at locus CA_C0175, position 198989 in the Clostridium acetobutylicum genome, coding for a predicted sugar phosphate isomerase, homolog of an eukaryotic glucokinase regulator (carbohydrate metabolism)
G is replaced with A at locus CA_C1300, position 1444099 in the Clostridium acetobutylicum genome, coding for an RNA polymerase sigma factor RPOD transcription and translation regulation)
C is replaced with T at locus CA_C2670, position 2787387 in the Clostridium acetobutylicum genome, coding for a Glu-tRNAGln amidotransferase subunit A (transcription and translation regulation)
C is replaced with T at locus CA_C3339, position 3512658 in the Clostridium acetobutylicum genome, coding for an ATPase component of an ABC transporter (two ATPase domains)
C is replaced with T at locus CA_C1610, position 1752341 in the Clostridium acetobutylicum genome, coding for a branched-chain amino acid permease (transporter).

The present invention also concerns a method for the production of 1,3-propanediol, comprising culturing a population of Clostridium acetobutylicum useful for the production of 1,3-propanediol (PDO) according to the invention in a culture medium comprising glycerine as sole source of carbon and recovering the 1,3-propanediol produced from the culture medium.

### DETAILED DESCRIPTION OF THE INVENTION

Population of Clostridium acetobutylicum Useful for the Production of 1,3-propanediol (PDO)

A population of Clostridium acetobutylicum useful for the production of 1,3-propanediol means one or more strains of Clostridium acetobutylicum genetically modified for the production of 1,3-propanediol from glycerine as sole source of carbon. Such strains are known in the art and disclosed, particularly, in applications WO200104324 and WO2008052595. The population according to the invention may be a combination of several strains, the majority of which comprising the mutations according to the invention, as well as a single strain, and particularly strain DG1 pSPD5 PD0001VE05c01, DG1 pSPD5 PD0001VE05c05 or DG1 pSPD5 PD0001VE05c07 deposited at CNCM under accession numbers I-4378, I-4379, I-4380 respectively, or strain DG1 pSPD5 PD0001VE05c08.

Mutations are changes of nucleotides in the strain genome, more particularly SNPs (“Single Nucleotide Polymorphisms”), identified when compared to the mother strain DG1 pSPD5 PD0001VT. Said strain is disclosed in WO200104324 and is derived from strain ATCC824 which genome sequence has been published (Nölling et al., 2001).

Mutations can occur in coding or non-coding sequences. These mutations can be synonymous wherein there is not modification of the corresponding amino acid or non-synonymous wherein the corresponding amino acid is altered. Synonymous mutations do not have any impact on the function of translated proteins, but may have an impact on the regulation of the corresponding genes or even of other genes, if the mutated sequence is located in a binding site for a regulator factor. Non-synonymous mutations may have an impact on the function of the translated protein as well as on regulation depending the nature of the mutated sequence.

The population of Clostridium acetobutylicum useful for the production of 1,3-propanediol may preferably comprise one of those deposited strains comprising additional modifications, at least one of the following modifications:
C replaced with T at locus CA_C0175, position 198989 in the Clostridium acetobutylicum genome, coding for a predicted sugar phosphate isomerase, homolog of an eukaryotic glucokinase regulator (carbohydrate metabolism)
G replaced with A at locus CA_C1300, position 1444099 in the Clostridium acetobutylicum genome, coding for an RNA polymerase sigma factor RPOD transcription and translation regulation)
C replaced with T at locus CA_C2670, position 2787387 in the Clostridium acetobutylicum genome, coding for a Glu-tRNAGln amidotransferase subunit A (transcription and translation regulation)
C replaced with T at locus CA_C3339, position 3512658 in the Clostridium acetobutylicum genome, coding for an ATPase component of an ABC transporter (two ATPase domains)
C replaced with T at locus CA_C1610, position 1752341 in the Clostridium acetobutylicum genome, coding for a branched-chain amino acid permease (transporter).

It may preferably comprise any combinations of these mutations, comprising 1, 2, 3, 4 or 5 of these mutations.

The population of strains of the invention is capable of growing on a medium comprising up to 120 g·L⁻¹ of glycerine and more particularly of industrial glycerine.

The strains of the population of the invention may be obtained using standard techniques of mutagenesis and/or gene replacement in Clostridium, such as disclosed in application WO2008040387 which contents are incorporated herein by reference.

The person skilled in the art may start from one of the strains disclosed in applications WO200104324 and WO2008052595 as well as use one of the strains c01, c05 or c07 deposited at CNCM under accession numbers I-4378, I-4379, I-4380 respectively, and introduce additional mutations.

In a preferred embodiment, the population of the invention comprises strain DG1 pSPD5 PD0001VE05c08, which mutations are identified in Table 1. The person skilled in the art knows how to introduce the mutations into a Clostridium strain to generate a strain similar to strain DG1 pSPD5 PD0001VE05c08, starting from one of strains DG1 pSPD5 PD0001VE05c01, DG1 pSPD5 PD0001VE05c05 or DG1 pSPD5 PD0001VE05c07, deposited at CNCM under accession numbers I-4378, I-4379, I-4380 respectively and using standard gene replacement and recombination techniques.

### Culture Medium Comprising Glycerine

An “appropriate culture medium” or a “culture medium” refers to a culture medium optimized for the growth and the diol-production of the Clostridium strains or population. The fermentation process is generally conducted in reactors with a synthetic, particularly inorganic, culture medium of known defined composition adapted to the Clostridium species used and containing glycerine.

The term “synthetic medium” means a culture medium comprising a chemically defined composition on which organisms are grown. In the culture medium of the present invention, glycerine is advantageously the single source of carbon.

The terms “glycerine” and ‘glycerol” are synonymous and used interchangeably in this invention to refer to the same molecule.

In a particular embodiment, glycerine is added to the medium in the form of glycerine composition comprising at least 50% of glycerine, preferably at least 85% of glycerine.

Advantageously, the glycerine used in the culture medium of the invention is industrial glycerine. “Industrial glycerine” means a glycerine product obtained from an industrial process without substantial purification. Industrial glycerine can also be designated as “raw glycerine”. Industrial glycerine comprises more than 70% of glycerine, preferably more than 80%, water and impurities such as mineral salts and fatty acids. The maximum content of glycerine in industrial glycerine is generally 90%, more generally about 85%.

Industrial processes form which industrial glycerine is obtained are, inter alia, manufacturing methods where fats and oils, particularly fats and oils of plant origin, are processed into industrial products such as detergent or lubricants. In such manufacturing methods, industrial glycerine is considered as a by-product.

In a particular embodiment, the industrial glycerine is a by-product from biodiesel production and comprises known impurities of glycerine obtained from biodiesel production, comprising about 80 to 85% of glycerine with salts, water and some other organic compounds such as fatty acids. Industrial glycerine obtained from biodiesel production has not been subjected to further purification steps.

Preferably, the culture medium comprises high concentrations of glycerine.

The terms “high glycerine content” or “high concentration of glycerine” means more than 90 g·L⁻¹ of glycerine in the culture medium. In preferred embodiments, the concentration is comprised between 90 and 200 g·L⁻¹ of glycerine, more particularly between 90 and 140 g/L of glycerine, preferably about 120 g·L⁻¹ of glycerine.

Preferably, the culture medium is a synthetic medium without addition of organic nitrogen.

Such culture media are disclosed in the art, particularly in PCT/EP2010/056078 filed on May 5, 2010 and PCT/EP2010/064825 filed on May 10, 2010, which contents are incorporated herein by reference.

### Culturing the Microorganisms

In the method of the invention, the production is advantageously done in a batch, fed-batch or continuous process. Culturing microorganisms at industrial scale for the production of 1,3-propanediol is known in the art, particularly disclosed in PCT/EP2010/056078 filed on May 5, 2010 and PCT/EP2010/064825 filed on May 10, 2010, which content are incorporated herein by reference.

1,3-propanediol Recovery

Methods for recovering and eventually purifying 1,3-propanediol from a fermentation medium are known to the skilled person. 1,3-propanediol may be isolated by distillation. In most embodiments, 1,3-propanediol is distilled from the fermentation medium with a by-product, such as acetate, and then further purified by known methods.

A particular purification method is disclosed in applications WO2009/068110 and WO 2010/037843, which content is incorporated herein by reference.

### FIGURES

FIG. 1 describes the evolution of 1,3-propanediol production and glycerine consumption of the population and clone c08 during the chemostat cultures from inoculation up to D=0.06 h⁻¹.

### EXAMPLES

### Example 1

### Isolation of Clones from the Evolved Population

Clone isolation was performed on agar plates starting from a growing flask culture of the population strain Clostridium acetobutylicum DG1 pSPD5 PD0001VE05. The synthetic media used for flask culture contained per liter of deionized water:glycerine, 30 g; KH₂PO₄, 0.5 g; K₂HPO₄, 0.5 g; MgSO₄, 7H₂O, 0.2 g; CoCl₂ 6H₂O, 0.01 g; acetic acid, 99.8%, 2.2 ml; NH₄Cl, 1.65 g; MOPS, 23.03 g, biotin, 0.16 mg; p-amino benzoic acid, 32 mg; FeSO₄, 7H₂O, 0.028 g; resazurin, 1 mg and cysteine, 0.5 g. The pH of the medium was adjusted to 6.5 with NH₄OH 6N.

Different media were used for isolation on agar plates:synthetic agar medium (the same as described above) with either commercial glycerine or raw glycerine and CGM (Clostridial Growth Medium) agar medium which contains per liter of deionized water:commercial or raw glycerine, 30 g; yeast extract, 5 g; KH₂PO₄, 0.75; K₂HPO₄, 0.75 g; MgSO₄, 7H₂O, 0.4 g; asparagine, 2 g; (NH₄)₂SO₄, 2 g; NaCl, 1 g; MnSO₄, H2O, 10 mg; FeSO₄, 7H₂O, 10 mg; MOPS, 23.03 g; resasurin, 1 mg and cysteine, 15 g. The pH of the medium was adjusted to 6.6 with NH₄OH 3N.

Cells were plated from a flask culture (Table 2) in four different ways:
on agar plates of synthetic medium with commercial glycerine;
on agar plates of synthetic medium with raw glycerine;
on agar plates of rich medium with commercial glycerine;
on agar plates of rich medium with raw glycerine.

Isolated clones were considered pure after three subsequent subcultures on agar plates. Pure clones were then transferred into liquid rich medium which contained either commercial or raw glycerine (Table 2). Subsequently, growing liquid cultures were conserved on glycerine 20% at −80° C. until further characterization.

Clones were then characterized in the following way:
Measurement of viability after conservation: evaluation of growth rate of cells on synthetic medium;
Evaluation of growth and metabolism: measurement of OD_{620 nm} during culture and PDO/glycerine yield on synthetic medium;
Genetic evaluation: PCR analysis to confirm the genotype of the strain;
Chemostat culture to compare the performances of isolated clones with those of the population (example 2);
gDNA extraction for sequence analysis of the clones (example 3).

TABLE 2
Synthetic agar media and liquid media used for
the isolation of 4 clones from the population.

| Clone | | Liquid media for clone culture |
|---|---|---|
| number | Agar media for isolation | before conservation |
| c01 | Synthetic medium with | Rich medium with commercial |
| | commercial glycerine | glycerine |
| c05 | Rich medium with raw | Rich medium with commercial |
| | glycerine | glycerine |
| c07 | Synthetic medium with | Rich medium with raw glycerine |
| | commercial glycerine | |
| c08 | Rich medium with | Rich medium with raw glycerine |
| | commercial glycerine | |

### Example 2

### Performances of Clone c08 in a Chemostat Culture with High Concentrations of Raw Glycerine

### Bacterial Strain:

Isolated clone of C. acetobutylicum strain DG1 pSPD5 PD0001VE05 (strain was 1/cured from pSOL1 2/transformed with plasmid pSPD5 harbouring dhaB1, dhaB2 and dhaT genes, ie 1,3-propanediol operon, and 3/evolved on high concentrations of raw glycerine). The isolation protocol was described in example 1.

### Culture Media:

The synthetic media used for clostridia batch cultivations contained per liter of deionized water: glycerine, 30 g; KH₂PO₄, 0.5 g; K₂HPO₄, 0.5 g; MgSO₄, 7H₂O, 0.2 g; CoCl₂ 6H₂O, 0.01 g; H₂SO₄, 0.1 ml; NH₄Cl, 1.5 g; biotin, 0.16 mg; p-amino benzoic acid, 32 mg and FeSO₄, 7H₂O, 0.028 g. The pH of the medium was adjusted to 6.3 with NH₄OH 3N. Commercial glycerine purchased from Sigma (purity 99.5%) was used for batch cultivation. The feed medium for continuous cultures contained per liter of tap water:raw glycerine, 105 g; KH₂PO₄, 0.5 g; K₂HPO₄, 0.5 g; MgSO₄, 7H₂O, 0.2 g; CoCl₂ 6H₂O, 0.026 g; NH₄Cl, 1.5 g; biotin, 0.16 mg; p-amino benzoic acid, 32 mg; FeSO₄, 7H₂O, 0.04 g; anti-foam, 0.05 ml; ZnSO₄, 7H₂O, 8 mg; CuCl₂, 2H₂O, 4 mg; MnSO₄, H₂O, 40 mg; H₃BO₃, 2 mg; Na₂MoO₄, 2H₂O, 0.8 mg. Medium pH was not adjusted in this case. Raw glycerine, from the transesterification process for biodiesel, was supplied by Novance (Venette, France) and had the following purity: glycerine 84.8% (w/w).

### Experimental Set-Up:

Continuous cultures were performed in a 51 bioreactor Tryton (Pierre Guerin, France) with a working volume of 2000 ml. The culture volume was kept constant at 2000 ml by automatic regulation of the culture level. Cultures were stirred at 200 RPM, the temperature was set to 35° C. and pH maintained constant at 6.5 by automatic addition of NH₄OH 5.5N. The POR measurement (mV) was controlled during the entire culture. To create anaerobic conditions, the sterilized medium in the vessel was flushed with sterile O₂-free nitrogen for one hour at 60° C. and flushed again until 35° C. was attained (flushing during 2 hours). The bioreactor gas outlet was protected from oxygen by a pyrogallol arrangement (Vasconcelos et al, 1994). After sterilisation the feed medium was also flushed with sterile O₂-free nitrogen until room temperature was attained and maintained under nitrogen at 200 mbar to avoid O₂ entry.

### Batch and Continuous Cultures Process:

The process used to evaluate has been described in patent application PCT/EP2010/056078 (example 2).

A culture growing in a 100 ml flask on synthetic medium (the same as described above for batch culture but with addition of acetic acid, 2.2 g·L⁻¹ and MOPS, 23.03 g·L⁻¹) taken at the end of exponential growth phase was used as inoculum (5% v/v).

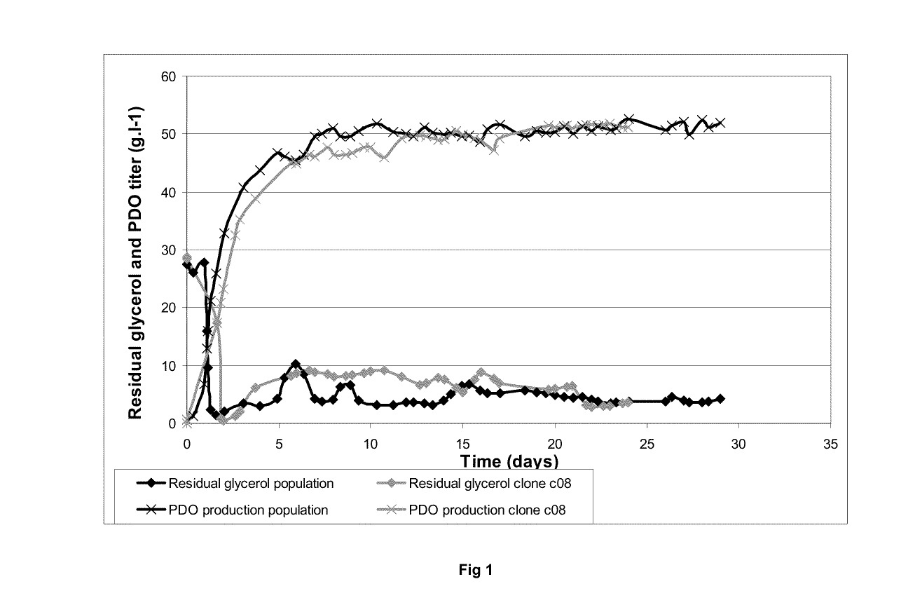

Cultures were first grown batchwise. At the early exponential growth phase we performed a pulse of commercial glycerine: For the pulse synthetic medium (the same as described for batch culture) with 105 g·L⁻¹ raw glycerine was added at a static flow rate during 3 hours (i.e. an addition of 18 g·L⁻¹ of glycerine). Then the growth continued batchwise and before the end of the exponential growth phase the continuous feeding started with a dilution rate of 0.025 h⁻¹: The feed medium contains 105 g·L⁻¹ of raw glycerine. 8-10 days after inoculation of the bioreactor and after 3 residence times the dilution rate was increased from 0.025 h⁻¹ to 0.060 h⁻¹ by different stages: Increase of 0.01 h⁻¹ units in 48 hours—no change for 24-hours—increase of 0.01 h⁻¹ units in 48 hours—no change for 24 hours—increase of 0.015 h⁻¹ unit in 48 hours. After that, stabilisation of the culture was followed by 1,3-propanediol production and glycerine consumption (FIG. 1) using the HPLC protocol described below. Particularly we waited until the concentration of residual glycerine was as low as possible.

The overall performances of c08 clone are presented in Table 3 and compared with performances of the population under the same conditions and with performances of the strain C. acetobutylicum DG1 pSPD5 PD0001VT such as described in Gonzalez-Pajuelo et al. (2005).

### Analytical Procedures:

Cell concentration was measured turbidimetrically at 620 nm and correlated with cell dry weight determined directly. Glycerine, 1,3-propanediol, ethanol, butanol, acetic and butyric acids concentrations were determined by HPLC analysis. Separation was performed on a Biorad Aminex HPX-87H column and detection was achieved by refractive index.

Operating conditions were as follows: mobile phase sulphuric acid 0.5 mM; flow rate 0.5 ml/min, temperature, 25° C.

TABLE 3
performances of the C. acetobutylicum DG1 pSPD5 population PD0001VE05 (mean data
from 4 chemostats), of clone c08 PD0001VE05c08. The feed medium contained 105 g · L⁻¹
of raw glycerine, dilution rate was 0.060 h⁻¹ and 0.025 h⁻¹. Values correspond to the
average of samples analyzed after at least 3 residences times at dilution rate of 0.060 h⁻¹.

| | | | Average and standard |
|---|---|---|---|
| | Average and | Average and | deviation of the strain |
| | standard deviation | standard deviation of | PD0001VT |
| | for clone c08 | the population | (Gonzalez-Pajuelo et |
| | PD0001VE05c08 | PD0001VE05 | al. 2005) |
| Feed glycerol (g · l⁻¹) | 105.49 +/− 1.07 | 104.21 +/− 1.36 | 58.54 |
| 1,3-propanediol (g · l⁻¹)) | 51.30 +/− 0.54 | 50.45 +/− 1.00 | 29.76 |
| Y_{1,3-PDO} (g · g⁻¹) | 0.50 +/− 0.01 | 0.53 +/− 0.01 | 0.50 |
| q_{1,3pdo} (q · l⁻¹ · h⁻¹) | 3.05 +/− 0.03 | 3.18 +/− 0.21 | 1.49 |
| Dilution rate (h⁻¹) | 0.059 +/− 0.001 | 0.063 +/− 0.004 | 0.05 |
| Residual glycerol (g · l⁻¹) | 3.72 +/− 1.62 | 4.82 +/− 1.82 | 0 |
| Biomass (g · l⁻¹) | 1.52 +/− 0.55 | 2.09 +/− 0.15 | 1.64 |
| Acetic acid (g · l⁻¹) | 2.67 +/− 0.26 | 2.09 +/− 0.27 | NI |
| Butyric acid (g · l⁻¹) | 10.53 +/− 0.38 | 10.98 +/− 0.37 | NI |

Y_{1,3-PDO}: PDO yield (g/g of glycerol consumed)
Q_{1,3-PDO}: PDO volumetric productivity
NI: no information. The PD0001VT strain can not grow in a medium lacking yeast extract.

These results show that the adapted population of C. acetobutylicum DG1 pSPD5 is able to grow on higher concentrations of industrial glycerine and thus exhibits a better titer and productivity of PDO on industrial glycerine, than the non adapted strain C. acetobutylicum DG1 pSPD5 PD0001VT from Gonzalez-Pajuelo et al. (2005) which can not grow in a medium lacking yeast extract.

### Example 3

### Genomic DNA Extraction

Genomic DNA from strains PD0001VT, PD0001VE05, PD0001VE05c01, PD0001VE05c05, PD0001VE05c07 and PD0001VE05c08 was extracted using Qiagen Genomic kit 500G (Qiagen, Inc., Valencia, Calif.). Briefly, cells were grown anaerobically respectively in rich or synthetic glycerine medium (as described in example 1 and 2) in penicillin vials (70 mL) to late exponential phase (A₆₂₀ 1.5 to 2.0). Strictly anaerobic conditions were maintained throughout cell lysis. Cells were collected and washed twice in SET buffer (25% sucrose, 0.05 M Tris-HCl, 0.05 M EDTA). Cell pellets were suspended in 11 mL B1 kit buffer with 44 μL RNase, 30 mg/mL lysozyme and 100 μg/mL proteinase K. The mixtures were incubated at 37° C. for 45 min, centrifuged and supernatants were used for DNA extraction according to the Qiagen DNA purification kit instructions. The DNAs were then suspended in 50 μL of 10 mM Tris-HCl (pH8.0).

### Sequencing Analysis

Genomes of the native DG1 pSPD5 PD0001VT strain and the evolved population DG1 pSPD5 PD0001VE05 were sequenced using the Roche GS FLX technology. The sequencing project was performed by Eurofins Genomics MWG/Operon (ZA de Courtabeauf-9 Avenue de la Laponie, 91978 Les Ulis Cedex) with for each strain 1 Long-Tag paired end libraries (8 Kb), generation of sequence and scaffolding of the contigs with GS FLX Titanium series chemistry using a half run (max. 600 000 reads, max 180 000-300 000 true paired end reads).

Isolated clones from the evolved population were sequenced using the comparative genomic sequencing (CGS) method developed by NimbleGen (Roche NimbleGen Inc. 500 S. Rosa Rd. Madison Wis. 53719). The CGS analysis was performed in two phases: in phase 1, regions of genomic difference were identified by a comparative hybridization of DNA of the native strain and the evolved clones. In phase 2, only the identified regions of genomic differences were sequenced so as to produce a set of fully characterized single nucleotide polymorphisms (SNPs).

### SNP Analysis

Bioinformatics and SNP analysis of the evolved population were performed by Eurofins Genomics MWG/Operon. For this analysis, the read sets of both strains were separately mapped to the Genbank reference sequence (Clostridium acetobutylicum ATCC 824 http://www.ncbi.nlm.nih.gov/nuccore/AE001437) using the software gsMapper (Roche 454, V2.3). Three SNPs files were delivered comparing DG1 pSPD5 PD0001VT to ATCC824, DG1 pSPD5 PD0001VE05 to ATCC824 and DG1 pSPD5 PD0001VT to DG1 pSPD5 PD0001VE05. Unique SNPs between the native and the evolved strains are presented below. Low coverage (<25) and low variant frequency (<85%) were removed resulting in 160 unique SNPs distributed in 17 families according to the KEGG database used for the family group annotations.

SNP analysis of the isolated clones was performed by NimbleGen (Roche). The SNP files were delivered comparing native DG1 pSPD5 PD0001VT to DG1 pSPD5 PD0001VE05c01, DG1 pSPD5 PD0001VE05c05, DG1 pSPD5 PD0001VE05c07 or DG1 pSPD5 PD0001VE05c08 using Genbank reference sequence (Clostridium acetobutylicum ATCC 824 http://www.ncbi.nlm.nih.gov/nuccore/AE001437).

The sequence results are presented in Table 1 which contains the following information:
RefStart the start position within the reference sequence, where the difference occurs
RefNuc the reference nucleotide sequence at the difference location
VarNuc the differing nucleotide sequence at the difference location
VarFreq the percentage of different reads versus total reads that fully span the difference location
Type Lists whether or not an SNP is found within an annotated gene, or between annotated genes. SNPs in genes are designated as coding. SNPs between genes are designated as intergenic
AA change categorizes coding SNPs base on whether or not they change the amino acid sequence of a protein. S indicates synonymous SNPs (no amino acid change). N indicates nonsynonymous SNPs (altered amino acid). FC (Frame-Change) indicates a modification in protein translation because of insertion or deletion of a nucleotide
ORIG_AA the amino acid associated with the reference sequence for the corresponding SNP position
SNP_AA the amino acid associated with the test sequence, for the corresponding SNP position
Locus Tag locus tag of the corresponding gene from Genbank
Function the function of the gene as described in Genbank
Family the family of the gene from KEGG

TABLE 1
Mutations between native and evolved strains. Mutations were first identified in the adapted population and then presence
of each mutation was verified in isolated clones (four last columns: Y for presence and N for absence of mutation).

| Ref | Ref | Var | Var | | AA | YRIG | SNP | Locus | | | | | | |
|---|---|---|---|---|---|---|---|---|---|---|---|---|---|---|
| Start | Nuc | Nuc | Freq | Type | cha. | AA | AA | Tag | Function | Family | c01 | c05 | c07 | C08 |
| 15594 | C | T | >99% | C | N | L | P | CA_C0009 | Uncharacterized | Hypothetical | Y | Y | Y | Y |
| | | | | | | | | | conserved protein, | proteins | | | | |
| | | | | | | | | | ortholog of YRXA | | | | | |
| | | | | | | | | | B. subtilis | | | | | |
| 21339 | G | A | >99% | I | | | | | I | I | Y | Y | Y | Y |
| 25354 | G | A | >99% | C | N | G | S | CA_C0017 | Seryl-tRNA synthetase | Transcription | Y | Y | Y | Y |
| | | | | | | | | | | translation | | | | |
| | | | | | | | | | | regulation | | | | |
| 29516 | G | A | >99% | C | N | G | D | CA_C0020 | MDR-type permease | Transporters | Y | Y | Y | Y |
| 31667 | C | T | >99% | C | N | A | V | CA_C0021 | Seryl-tRNA synthetase | Transcription | Y | Y | Y | Y |
| | | | | | | | | | (serine-tRNA ligase) | translation | | | | |
| | | | | | | | | | | regulation | | | | |
| 35547 | G | A | >99% | C | S | R | R | CA_C0024 | Membrane protein, | Membrane | Y | Y | Y | Y |
| | | | | | | | | | related to | proteins | | | | |
| | | | | | | | | | Actinobacillus protein | | | | | |
| | | | | | | | | | (1944168) | | | | | |
| 43029 | C | T | >99% | C | N | A | V | CA_C0032 | Transcriptional | Transcription | Y | Y | Y | Y |
| | | | | | | | | | regulator TetR/AcrR | translation | | | | |
| | | | | | | | | | family | regulation | | | | |
| 52503 | A | G | >99% | C | S | A | A | CA_C0039 | DNA segregation | Cell division | Y | Y | Y | Y |
| | | | | | | | | | ATPase FtsK/SpoIIIE | | | | | |
| | | | | | | | | | family protein, | | | | | |
| | | | | | | | | | contains FHA domain | | | | | |
| 76769 | C | T | >99% | C | S | F | F | CA_C0066 | ABC transporter, ATP- | Transporters | Y | Y | Y | Y |
| | | | | | | | | | binding protein | | | | | |
| 143022 | G | A | >99% | C | S | S | S | CA_C0135 | Hypothetical protein, | Hypothetical | Y | Y | Y | Y |
| | | | | | | | | | CF-23 family | proteins | | | | |
| 198989 | C | T | >99% | C | N | T | I | CA_C0175 | Predicted sugar | Carbohydrate | Y | Y | Y | Y |
| | | | | | | | | | phosphate isomerase, | metabolism | | | | |
| | | | | | | | | | homolog of eucaryotic | | | | | |
| | | | | | | | | | glucokinase regulator | | | | | |
| 219199 | C | T | >99% | C | N | M | I | CA_C0193 | Uncharacterized | Membrane | Y | Y | Y | Y |
| | | | | | | | | | conserved membrane | proteins | | | | |
| | | | | | | | | | protein, affecting | | | | | |
| | | | | | | | | | LPS biosynthesis | | | | | |
| 220222 | T | C | >99% | C | N | N | S | CA_C0194 | Glycosyltransferase | Carbohydrate | Y | Y | Y | Y |
| | | | | | | | | | involved in cell wall | metabolism | | | | |
| | | | | | | | | | biogenesis | | | | | |
| 265152 | G | A | >99% | C | N | G | S | CA_C0234 | PTS system, fructoso- | Carbohydrate | Y | Y | Y | Y |
| | | | | | | | | | specific IIBC | metabolism | | | | |
| | | | | | | | | | component | | | | | |
| 286308 | G | A | >99% | C | N | A | T | CA_C0256 | Nitrogenase | Energy | Y | Y | Y | Y |
| | | | | | | | | | molybdenum-iron | metabolism | | | | |
| | | | | | | | | | protein, alpha chain | | | | | |
| | | | | | | | | | (nitrogenase component | | | | | |
| | | | | | | | | | I) gene nifD | | | | | |
| 347024 | G | A | >99% | C | N | A | T | CA_C0291 | FUSION: methionine | Amino acid | Y | Y | Y | Y |
| | | | | | | | | | synthase I (cobalamin | metabolism | | | | |
| | | | | | | | | | dependent) and 5,10 | | | | | |
| | | | | | | | | | methylenetetrahydro- | | | | | |
| | | | | | | | | | folate reductase | | | | | |
| 364263 | A | G | >99% | I | | | | | I | I | Y | Y | Y | Y |
| 454074 | G | A | >99% | C | N | E | K | CA_C0390 | Cystathionine gamma- | Amino acid | Y | Y | Y | Y |
| | | | | | | | | | synthase | metabolism | | | | |
| 541268 | C | T | >99% | C | N | T | I | CA_C0471 | GrpE protein HSP-70 | Transcription | Y | Y | Y | Y |
| | | | | | | | | | cofactor | translation | | | | |
| | | | | | | | | | | regulation | | | | |
| 601197 | A | G | >99% | I | | | | | I | I | Y | Y | Y | Y |
| 621074 | C | T | >99% | C | N | P | L | CA_C0534 | Phosphoenolpyruvate | Carbohydrate | Y | Y | Y | Y |
| | | | | | | | | | synthase (gene pps) | metabolism | | | | |
| 656050 | T | C | >99% | C | N | S | P | CA_C0566 | Malate dehydrogenase | Energy | Y | Y | Y | Y |
| | | | | | | | | | | metabolism | | | | |
| 668495 | A | G | >99% | C | S | I | I | CA_C0578 | Cobalamine-dependent | Amino acid | Y | Y | Y | Y |
| | | | | | | | | | methionine synthase I | metabolism | | | | |
| | | | | | | | | | (methyltransferase and | | | | | |
| | | | | | | | | | cobalamine-binding | | | | | |
| | | | | | | | | | domain) | | | | | |
| 723431 | — | A | >99% | I | | | | | I | I | N | N | N | N |
| 794841 | C | T | >99% | C | N | T | I | CA_C0688 | 1-acyl-sn-glycerine-3- | Glycerine | Y | Y | Y | Y |
| | | | | | | | | | phosphate acyltrans- | metabolism | | | | |
| | | | | | | | | | ferase | | | | | |
| 817479 | C | T | >99% | C | N | A | V | CA_C0706 | Endo-1,4-beta | Cellulase | Y | Y | Y | Y |
| | | | | | | | | | glucanase (fused to | | | | | |
| | | | | | | | | | two ricin-B-like | | | | | |
| | | | | | | | | | domains) | | | | | |
| 819542 | C | T | >99% | C | N | P | L | CA_C0707 | RNA polymerase sigma- | Transcription | Y | Y | Y | Y |
| | | | | | | | | | 54 factor | translation | | | | |
| | | | | | | | | | | regulation | | | | |
| 951584 | A | G | >99% | C | N | I | V | CA_C0823 | Predicted membrane | Membrane | Y | Y | Y | Y |
| | | | | | | | | | protein | proteins | | | | |
| 977563 | G | A | >99% | I | | | | | I | I | Y | Y | Y | Y |
| 978671 | C | T | >99% | C | S | C | C | CA_C0850 | Nitroreductase family | Energy | Y | Y | Y | Y |
| | | | | | | | | | protein | metabolism | | | | |
| 991021 | T | C | >99% | C | N | V | A | CA_C0861 | ABC-type multidrug | Transporters | Y | Y | Y | Y |
| | | | | | | | | | transport system, | | | | | |
| | | | | | | | | | ATPase component | | | | | |
| 991449 | G | A | >99% | C | N | G | R | CA_C0861 | ABC-type multidrug | Transporters | Y | Y | Y | Y |
| | | | | | | | | | transport system, | | | | | |
| | | | | | | | | | ATPase component | | | | | |
| 1019710 | G | A | >99% | C | N | V | . | CA_C0888 | Phosphoglycerine | Glycerine | Y | Y | Y | Y |
| | | | | | | | | | transferase MdoB | metabolism | | | | |
| | | | | | | | | | related protein, | | | | | |
| | | | | | | | | | alkaline phosphatase | | | | | |
| | | | | | | | | | superfamily | | | | | |
| 1068817 | T | C | >99% | C | N | L | S | CA_C0925 | TPR-repeat-containing | Hypothetical | Y | Y | Y | Y |
| | | | | | | | | | protein | proteins | | | | |
| 1113238 | G | A | >99% | C | N | N | S | CA_C0967 | Probably membrane | Membrane | Y | Y | Y | Y |
| | | | | | | | | | protein | proteins | | | | |
| 1223725 | G | A | >99% | C | N | A | T | CA_C1072 | Fe—S | Energy | Y | Y | Y | Y |
| | | | | | | | | | oxidoreductase | metabolism | | | | |
| 1254865 | T | A | >99% | C | N | Y | N | CA_C1086 | Transcriptional | Transcription | Y | Y | Y | Y |
| | | | | | | | | | regulators of | translation | | | | |
| | | | | | | | | | NagC/XylR family | regulation | | | | |
| 1299105 | A | G | >99% | C | N | M | T | CA_C1133 | Phage related protein, | Hypothetical | Y | Y | Y | Y |
| | | | | | | | | | YonE B. subtilis | proteins | | | | |
| | | | | | | | | | homolog | | | | | |
| 1309504 | C | T | >99% | C | N | M | I | CA_C1143 | Exodeoxyribonuclease | Cell division | Y | Y | Y | Y |
| | | | | | | | | | V, Alpha subunit, RecD | | | | | |
| 1324897 | A | G | >99% | C | S | P | P | CA_C1166 | Hypothetical protein | Hypothetical | Y | Y | Y | Y |
| | | | | | | | | | | proteins | | | | |
| 1366058 | T | A | >99% | C | N | N | K | CA_C1223 | DNA Polymerase III | Transcription | Y | Y | Y | Y |
| | | | | | | | | | Alpha chain (dnaE) | translation | | | | |
| | | | | | | | | | | regulation | | | | |
| 1424502 | A | G | >99% | C | N | Y | C | CA_C1280 | Transcriptional | Transcription | Y | Y | Y | Y |
| | | | | | | | | | regulator of heat | translation | | | | |
| | | | | | | | | | shock genes, HrcA | regulation | | | | |
| 1444099 | G | A | >99% | C | N | G | R | CA_C1300 | RNA polymerase sigma | Transcription | Y | Y | Y | Y |
| | | | | | | | | | factor RPOD | translation | | | | |
| | | | | | | | | | | regulation | | | | |
| 1540446 | A | G | >99% | C | S | L | L | CA_C1396 | Phosphoribosylamine- | Nucleic acid | Y | Y | Y | Y |
| | | | | | | | | | glycine ligase | metabolism | | | | |
| 1554592 | A | G | >99% | C | N | K | R | CA_C1408 | Phospho-beta-gluco- | Carbohydrate | Y | Y | Y | Y |
| | | | | | | | | | sidase | metabolism | | | | |
| 1644651 | A | — | >99% | I | | | | | I | I | Y | Y | Y | Y |
| 1770126 | A | G | >99% | C | N | I | V | CA_C1628 | DNA gyrase A subunit | Transcription | Y | Y | Y | Y |
| | | | | | | | | | | translation | | | | |
| | | | | | | | | | | regulation | | | | |
| 1778678 | G | A | >99% | C | N | A | T | CA_C1636 | Uncharacterized | Hypothetical | Y | Y | Y | Y |
| | | | | | | | | | protein, homolog of | proteins | | | | |
| | | | | | | | | | B. firmus (2654481) | | | | | |
| 1805186 | G | A | >99% | C | N | V | I | CA_C1661 | Predicted secreted | Nucleic acid | Y | Y | Y | Y |
| | | | | | | | | | nucleic acid binding | metabolism | | | | |
| | | | | | | | | | protein | | | | | |
| 1821699 | G | A | >99% | C | N | A | T | CA_C1673 | Large subunit of NADH- | Energy | Y | Y | Y | Y |
| | | | | | | | | | dependent glutamate | metabolism | | | | |
| | | | | | | | | | synthase | | | | | |
| 1916660 | A | G | >99% | C | N | N | S | CA_C1771 | Uncharacterized | Hypothetical | Y | Y | Y | Y |
| | | | | | | | | | protein, ykrI | proteins | | | | |
| | | | | | | | | | B. subtilis homolog | | | | | |
| 1948050 | C | T | >99% | I | | | | | I | I | Y | Y | Y | Y |
| 2037205 | G | A | >99% | C | S | C | C | CA_C1886 | Uncharacterized phage | Hypothetical | Y | Y | Y | Y |
| | | | | | | | | | related protein | proteins | | | | |
| 2114483 | A | G | >99% | C | N | V | A | CA_C2003 | Predicted permease | Transporters | Y | Y | Y | Y |
| 2123888 | T | C | >99% | C | S | L | L | CA_C2010 | Predicted Fe—S | Energy | Y | Y | Y | Y |
| | | | | | | | | | oxidoreductase | metabolism | | | | |
| 2171503 | C | T | >99% | C | N | D | N | CA_C2068 | Sporulation factor | Sporulation | Y | Y | Y | Y |
| | | | | | | | | | spoIIM, uncharacterized | | | | | |
| | | | | | | | | | membrane protein | | | | | |
| 2231570 | C | — | >99% | C | FC | | | CA_C2137 | Cation transport | Transporters | N | N | N | Y |
| | | | | | | | | | P-type ATPase | | | | | |
| 2294764 | G | A | >99% | C | N | T | I | CA_C2201 | Hypothetical protein | Hypothetical | Y | Y | Y | Y |
| | | | | | | | | | | proteins | | | | |
| 2299326 | C | G | >99% | C | N | S | T | CA_C2205 | Flagellar hook- | Cell motility | Y | Y | Y | Y |
| | | | | | | | | | associated protein | | | | | |
| | | | | | | | | | FliD | | | | | |
| 2307214 | C | T | >99% | C | N | G | R | CA_C2215 | Flagellar switch | Cell motility | Y | N | Y | Y |
| | | | | | | | | | protein FliY, contains | | | | | |
| | | | | | | | | | CheC-like domain | | | | | |
| 2342826 | G | C | >99% | C | N | P | A | CA_C2247 | Site-specific | Transcription | Y | Y | Y | Y |
| | | | | | | | | | recombinase, DNA | translation | | | | |
| | | | | | | | | | invertase Pin homolog | regulation | | | | |
| 2392178 | C | T | >99% | C | N | V | . | CA_C2288 | Acyl-protein | Lipid | Y | Y | Y | Y |
| | | | | | | | | | synthetase, luxE | metabolism | | | | |
| 2450006 | C | T | >99% | C | S | P | P | CA_C2340 | DNA mismatch repair | Transcription | Y | Y | Y | Y |
| | | | | | | | | | protein mutS, YSHD | translation | | | | |
| | | | | | | | | | B. subtilis ortholog | regulation | | | | |
| 2477825 | C | T | >99% | C | S | S | S | CA_C2367 | Uncharacterized | Cell adhesion | Y | Y | Y | Y |
| | | | | | | | | | protein containing | | | | | |
| | | | | | | | | | predicted cell | | | | | |
| | | | | | | | | | adhesion domain and | | | | | |
| | | | | | | | | | ChW-repeats | | | | | |
| 2493211 | T | C | >99% | C | S | H | H | CA_C2385 | Hypothetical protein | Hypothetical | Y | Y | Y | Y |
| | | | | | | | | | | proteins | | | | |
| 2595349 | G | A | >99% | C | N | A | V | CA_C2486 | Transcriptional | Transcription | Y | Y | Y | Y |
| | | | | | | | | | regulator, MarR/EmrR | translation | | | | |
| | | | | | | | | | family | regulation | | | | |
| 2693354 | C | T | >99% | C | N | E | K | CA_C2588 | Glycosyltransferase | Carbohydrate | Y | Y | Y | Y |
| | | | | | | | | | | metabolism | | | | |
| 2787387 | C | T | >99% | C | N | M | I | CA_C2670 | Glu-tRNAGln | Transcription | Y | Y | Y | Y |
| | | | | | | | | | amidotransferase | translation | | | | |
| | | | | | | | | | subunit A | regulation | | | | |
| 2833384 | T | C | >99% | C | N | I | V | CA_C2709 | Electron transfer | Energy | Y | Y | Y | Y |
| | | | | | | | | | flavoprotein alpha- | metabolism | | | | |
| | | | | | | | | | subunit | | | | | |
| 2836979 | G | A | >99% | C | N | A | V | CA_C2713 | AT-rich DNA-binding | Transcription | Y | Y | Y | Y |
| | | | | | | | | | protein | translation | | | | |
| | | | | | | | | | | regulation | | | | |
| 2901642 | C | T | >99% | C | N | V | . | CA_C2770 | Amino acid transporter | Transporters | Y | Y | Y | Y |
| 2969858 | G | A | >99% | C | N | M | I | CA_C2838 | Predicted nucleotide- | Transcription | Y | Y | Y | Y |
| | | | | | | | | | binding protein, YjeE | translation | | | | |
| | | | | | | | | | family | regulation | | | | |
| 3001642 | G | A | >99% | C | S | L | L | CA_C2867 | FoF1-type ATP synthase | Energy | Y | Y | Y | Y |
| | | | | | | | | | alpha subunit | metabolism | | | | |
| 3032956 | T | C | >99% | C | N | H | R | CA_C2898 | Stage II sporulation | Sporulation | Y | Y | Y | Y |
| | | | | | | | | | protein R | | | | | |
| 3140918 | T | C | >99% | I | | | | | I | I | Y | Y | Y | Y |
| 3174743 | G | A | >99% | C | S | D | D | CA_C3032 | Galactose mutarotase | Carbohydrate | Y | N | Y | Y |
| | | | | | | | | | related enzyme | metabolism | | | | |
| 3251276 | G | C | >99% | C | N | T | S | CA_C3099 | Pseudouridylate | Nucleic acid | Y | Y | Y | Y |
| | | | | | | | | | synthase, TRUA | metabolism | | | | |
| 3337937 | G | — | >99% | I | | | | | I | I | N | N | N | N |
| 3392124 | G | A | >99% | C | N | G | R | CA_C3242 | Uncharacterized | Energy | Y | Y | Y | Y |
| | | | | | | | | | Fe—S protein, | metabolism | | | | |
| | | | | | | | | | PflX (pyruvate formate | | | | | |
| | | | | | | | | | lyase activating | | | | | |
| | | | | | | | | | protein) homolog | | | | | |
| 3462380 | C | T | >99% | C | S | N | N | CA_C3297 | D-alanyl-D-alanine | Hypothetical | Y | Y | Y | Y |
| | | | | | | | | | carboxypeptidase | proteins | | | | |
| | | | | | | | | | family hydrolase, | | | | | |
| | | | | | | | | | YODJ B. subtilis | | | | | |
| | | | | | | | | | ortholog | | | | | |
| 3509372 | C | T | >99% | C | S | E | E | CA_C3335 | Short-chain alcohol | Energy | Y | Y | Y | Y |
| | | | | | | | | | dehydrogenase family | metabolism | | | | |
| | | | | | | | | | enzyme | | | | | |
| 3512658 | C | T | >99% | C | S | Y | Y | CA_C3339 | ATPase component of | Transporters | Y | Y | Y | Y |
| | | | | | | | | | ABC transporter (two | | | | | |
| | | | | | | | | | ATPase domains) | | | | | |
| 3518240 | T | C | >99% | C | S | Y | Y | CA_C3345 | Transcriptional | Transcription | Y | Y | Y | Y |
| | | | | | | | | | regulator, AcrR family | translation | | | | |
| | | | | | | | | | | regulation | | | | |
| 3541557 | T | C | >99% | C | N | I | V | CA_C3363 | Hypothetical protein | Hypothetical | Y | Y | Y | Y |
| | | | | | | | | | | proteins | | | | |
| 3565291 | C | T | >99% | C | N | T | I | CA_C3387 | Pectate lyase | Cellulase | Y | Y | Y | Y |
| 3576865 | T | C | >99% | C | N | H | R | CA_C3392 | NADH-dependent | Energy | Y | Y | Y | Y |
| | | | | | | | | | butanol dehydrogenase | metabolism | | | | |
| 3583724 | C | T | >99% | I | | | | | I | I | Y | Y | Y | Y |
| 3608511 | C | T | >99% | C | S | S | S | CA_C3422 | Sugar: proton symporter | Transporters | Y | Y | Y | Y |
| | | | | | | | | | (possible xylulose) | | | | | |
| 3614985 | C | T | >99% | C | S | K | K | CA_C3428 | 6Fe—6S prismane | Energy | Y | Y | Y | Y |
| | | | | | | | | | cluster-containing | metabolism | | | | |
| | | | | | | | | | protein | | | | | |
| 3674358 | T | C | >99% | I | | | | | I | I | Y | Y | Y | Y |
| 3707038 | T | C | >99% | C | S | L | L | CA_C3510 | Membrane associated | Membrane | Y | Y | Y | Y |
| | | | | | | | | | methyl-accepting | proteins | | | | |
| | | | | | | | | | chemotaxis protein | | | | | |
| | | | | | | | | | (with HAMP domain) | | | | | |
| 3747653 | G | A | >99% | C | N | A | V | CA_C3551 | Na+ ABC transporter | Transporters | Y | Y | Y | Y |
| | | | | | | | | | (ATP-binding protein), | | | | | |
| | | | | | | | | | NATA | | | | | |
| 3821135 | C | T | >99% | C | S | N | N | CA_C3617 | Uncharacterized | Hypothetical | Y | Y | Y | Y |
| | | | | | | | | | membrane protein, | proteins | | | | |
| | | | | | | | | | YHAG B. subtilis | | | | | |
| | | | | | | | | | homolog | | | | | |
| 3850220 | A | G | >99% | C | N | I | T | CA_C3650 | HD-GYP domain | Proteases/ | Y | Y | Y | Y |
| | | | | | | | | | containing protein | Peptidases | | | | |
| 3921509 | C | T | >99% | C | N | V | I | CA_C3716 | Lon-like ATP-dependent | Proteases/ | Y | Y | Y | Y |
| | | | | | | | | | protease | Peptidases | | | | |
| 239312 | G | A | 98% | C | N | E | K | CA_C0214 | Endoglucanase, | Cellulase | Y | Y | Y | Y |
| | | | | | | | | | aminopeptidase M42 | | | | | |
| | | | | | | | | | family | | | | | |
| 244251 | C | T | 98% | I | | | | | I | I | Y | Y | Y | Y |
| 2410308 | G | A | 98% | C | S | L | L | CA_C2306 | Sporulation-specific | Sporulation | Y | Y | Y | Y |
| | | | | | | | | | sigma factor F | | | | | |
| 3656844 | G | A | 98% | C | N | A | T | CA_C3459 | Homolog of cell | Cell division | Y | Y | Y | Y |
| | | | | | | | | | division GTPase FtsZ, | | | | | |
| | | | | | | | | | diverged | | | | | |
| 3823060 | A | G | 98% | C | N | V | A | CA_C3620 | Amino acid (probably | Transporters | Y | Y | Y | Y |
| | | | | | | | | | glutamine) ABC | | | | | |
| | | | | | | | | | transporter, | | | | | |
| | | | | | | | | | periplasmic binding | | | | | |
| | | | | | | | | | protein component | | | | | |
| 3838496 | C | T | 98% | C | N | G | R | CA_C3637 | Oligopeptide ABC | Transporters | N | N | N | N |
| | | | | | | | | | transporter, permease | | | | | |
| | | | | | | | | | component | | | | | |
| 914 | G | A | 97% | C | N | G | R | CA_C0001 | DNA replication | Cell division | Y | Y | Y | Y |
| | | | | | | | | | initiator protein, | | | | | |
| | | | | | | | | | ATPase | | | | | |
| 27240 | C | T | 97% | C | N | R | Q | CA_C0019 | Transcriptional | Transcription | N | Y | N | Y |
| | | | | | | | | | regulator, AcrR family | translation | | | | |
| | | | | | | | | | | regulation | | | | |
| 36341 | G | A | 97% | C | N | D | N | CA_C0025 | Deoxycytidine | Nucleic acid | N | Y | N | N |
| | | | | | | | | | triphosphate deaminase | metabolism | | | | |
| 299266 | G | A | 97% | C | N | D | N | CA_C0267 | L-lactate dehydrogenase | Energy | Y | N | Y | Y |
| | | | | | | | | | | metabolism | | | | |
| 376886 | G | A | 97% | C | S | K | K | CA_C0322 | Sensory protein, | Transcription | Y | Y | Y | Y |
| | | | | | | | | | containing EAL-domain | translation | | | | |
| | | | | | | | | | | regulation | | | | |
| 474915 | G | A | 97% | C | N | G | D | CA_C0408 | DNA segregation | Cell division | Y | Y | Y | Y |
| | | | | | | | | | ATP-ase FtsK/SpoIIIE | | | | | |
| | | | | | | | | | (three ATPases), | | | | | |
| | | | | | | | | | contains FHA domain | | | | | |
| 599925 | G | A | 97% | C | N | R | K | CA_C0519 | Dihydroorotase | Nucleic acid | Y | Y | Y | Y |
| | | | | | | | | | | metabolism | | | | |
| 723433 | G | A | 97% | I | | | | | I | I | N | N | N | N |
| 723434 | GT | — | 97% | I | | | | | I | I | N | N | N | N |
| 723871 | A | G | 97% | C | N | I | M | CA_C0622 | Polyphosphate kinase | Energy | Y | Y | Y | Y |
| | | | | | | | | | | metabolism | | | | |
| 809008 | C | T | 97% | C | S | L | L | CA_C0699 | Spore photoproduct | Sporulation | Y | Y | Y | Y |
| | | | | | | | | | lyase, splB | | | | | |
| 846466 | G | T | 97% | C | N | A | S | CA_C0731 | FUSION: Nucleoside- | Nucleic acid | Y | Y | Y | Y |
| | | | | | | | | | diphosphate-sugar | metabolism | | | | |
| | | | | | | | | | epimerase and GAF | | | | | |
| | | | | | | | | | domain | | | | | |
| 1717948 | G | A | 97% | C | N | V | I | CA_C1572 | Fructose-1,6-bisphos- | Carbohydrate | Y | Y | Y | Y |
| | | | | | | | | | phatase (YYDE | metabolism | | | | |
| | | | | | | | | | B. subtils ortholog) | | | | | |
| 2004797 | C | T | 97% | C | N | S | N | CA_C1852 | Magnesium and cobalt | Transporters | Y | Y | Y | Y |
| | | | | | | | | | transport protein | | | | | |
| 2134058 | G | A | 97% | C | S | A | A | CA_C2020 | Molybdopterin | Energy | Y | Y | Y | Y |
| | | | | | | | | | bioSthesis enzyme, | metabolism | | | | |
| | | | | | | | | | MoeA, fused to | | | | | |
| | | | | | | | | | molibdopterin-binding | | | | | |
| | | | | | | | | | domain | | | | | |
| 2331746 | G | A | 97% | C | N | G | R | CA_C2237 | ADP-glucose | Lipid | Y | Y | Y | Y |
| | | | | | | | | | pyrophosphorylase | metabolism | | | | |
| 2391588 | G | A | 97% | C | N | P | L | CA_C2288 | Acyl-protein Sthetase, | Lipid | Y | Y | Y | Y |
| | | | | | | | | | luxE | metabolism | | | | |
| 2452705 | C | T | 97% | C | N | C | Y | CA_C2341 | Collagenase family | Proteases/ | Y | Y | Y | Y |
| | | | | | | | | | protease | Peptidases | | | | |
| 2739459 | T | C | 97% | C | N | I | V | CA_C2630 | Uncharaterized | Hypothetical | Y | Y | Y | Y |
| | | | | | | | | | conserved protein, | proteins | | | | |
| | | | | | | | | | YOME B. subtilis | | | | | |
| | | | | | | | | | ortholog | | | | | |
| 2775979 | C | T | 97% | C | N | A | T | CA_C2660 | Pyruvate carboxylase, | Carbohydrate | Y | Y | Y | Y |
| | | | | | | | | | PYKA | metabolism | | | | |
| 2813985 | G | — | 97% | I | | | | | I | I | N | N | N | N |
| 3082247 | C | T | 97% | C | N | L | F | CA_C2948 | ATPase components of | Transporters | Y | Y | Y | Y |
| | | | | | | | | | ABC transporter with | | | | | |
| | | | | | | | | | duplicated ATPase | | | | | |
| | | | | | | | | | domains (second domain | | | | | |
| | | | | | | | | | is inactivated) | | | | | |
| 3242900 | G | C | 97% | C | N | V | L | CA_C3088 | NtrC family | Transcription | Y | N | N | Y |
| | | | | | | | | | transcriptional | translation | | | | |
| | | | | | | | | | regulator, ATPase | regulation | | | | |
| | | | | | | | | | domain fused | | | | | |
| | | | | | | | | | to two PAS domains | | | | | |
| 3442855 | T | C | 97% | C | N | M | V | CA_C3282 | ABC-type | Transporters | Y | Y | Y | Y |
| | | | | | | | | | multidrug/protein/ | | | | | |
| | | | | | | | | | lipid transport system, | | | | | |
| | | | | | | | | | ATPase component | | | | | |
| 3498584 | C | T | 97% | C | N | L | F | CA_C3327 | Amino acid ABC-type | Transporters | Y | Y | Y | Y |
| | | | | | | | | | transporter, ATPase | | | | | |
| | | | | | | | | | component | | | | | |
| 3643224 | G | A | 97% | C | S | L | L | CA_C3447 | Protein-disulfide | Sporulation | Y | Y | Y | Y |
| | | | | | | | | | isomerases DsbC/DsbG | | | | | |
| 3663477 | — | T | 97% | C | FC | | | CA_C3464 | Uncharacterized | Hypothetical | N | N | N | N |
| | | | | | | | | | conserved protein | proteins | | | | |
| | | | | | | | | | (fragment) | | | | | |
| 204202 | G | A | 96% | C | N | G | E | CA_C0180 | Oligopeptide ABC | Transporters | Y | Y | Y | Y |
| | | | | | | | | | transporter, ATP- | | | | | |
| | | | | | | | | | binding protein | | | | | |
| 803682 | C | T | 96% | C | N | T | I | CA_C0695 | Altronate | Carbohydrate | Y | Y | Y | Y |
| | | | | | | | | | oxidoreductase | metabolism | | | | |
| 892875 | G | A | 96% | C | N | M | I | CA_C0770 | Glycerine uptake | Glycerine | Y | Y | Y | Y |
| | | | | | | | | | facilitator protein, | metabolism | | | | |
| | | | | | | | | | permease | | | | | |
| 1009389 | C | T | 96% | C | N | P | S | CA_C0879 | ABC-type polar amino | Transporters | Y | Y | Y | Y |
| | | | | | | | | | acid transport system, | | | | | |
| | | | | | | | | | ATPase component | | | | | |
| 1690355 | C | T | 96% | C | S | G | G | CA_C1546 | Pyrimidine-nucleoside | Nucleic acid | Y | Y | Y | Y |
| | | | | | | | | | phosphorylase | metabolism | | | | |
| 1752341 | C | T | 96% | C | N | G | R | CA_C1610 | Branched-chain amino | Transporters | Y | Y | Y | Y |
| | | | | | | | | | acid permease | | | | | |
| 3217481 | A | C | 96% | C | S | L | L | CA_C3067 | Predicted membrane | Membrane | Y | Y | Y | Y |
| | | | | | | | | | protein | proteins | | | | |
| 3238489 | T | C | 96% | C | S | S | S | CA_C3086 | Protein containing | Cell adhesion | Y | Y | Y | Y |
| | | | | | | | | | cell adhesion domain | | | | | |
| 447460 | A | — | 95% | I | | | | | I | I | N | N | N | N |
| 670931 | G | A | 95% | C | S | N | N | CA_C0578 | Cobalamine-dependent | Amino acid | N | Y | Y | Y |
| | | | | | | | | | methionine synthase I | metabolism | | | | |
| | | | | | | | | | (methyltransferase and | | | | | |
| | | | | | | | | | cobalamine-binding | | | | | |
| | | | | | | | | | domain) | | | | | |
| 994575 | G | A | 95% | C | N | A | T | CA_C0864 | Histidine kinase-like | Transcription | Y | Y | Y | Y |
| | | | | | | | | | ATPase | translation | | | | |
| | | | | | | | | | | regulation | | | | |
| 3657101 | A | — | 95% | C | FC | | | CA_C3459 | Homolog of cell | Cell division | N | N | N | N |
| | | | | | | | | | division GTPase FtsZ, | | | | | |
| | | | | | | | | | diverged | | | | | |
| 1142263 | T | — | 94% | C | FC | | | CA_C0995 | Predicted membrane | Membrane | N | N | N | N |
| | | | | | | | | | protein | proteins | | | | |
| 1823156 | G | A | 94% | C | S | E | E | CA_C1674 | Small subunit of | Amino acid | Y | Y | Y | Y |
| | | | | | | | | | NADPH-dependent | metabolism | | | | |
| | | | | | | | | | glutamate synthase | | | | | |
| 1989117 | C | T | 94% | C | N | R | K | CA_C1837 | Mismatch repair | Transcription | Y | Y | Y | Y |
| | | | | | | | | | protein MutS, ATPase | translation | | | | |
| | | | | | | | | | | regulation | | | | |
| 3481651 | G | A | 94% | C | S | S | S | CA_C3311 | TPR-repeat domain | Carbohydrate | Y | Y | Y | Y |
| | | | | | | | | | fused to glycosyltrans- | metabolism | | | | |
| | | | | | | | | | ferase | | | | | |
| 126942 | G | A | 93% | C | N | E | K | CA_C0116 | Carbone-monoxide | Energy | Y | Y | Y | Y |
| | | | | | | | | | dehydrogenase, beta | metabolism | | | | |
| | | | | | | | | | chain | | | | | |
| 302716 | — | T | 93% | C | FC | | | CA_C0270 | Hypothetical protein | Hypothetical | N | N | N | N |
| | | | | | | | | | | proteins | | | | |
| 2551103 | G | A | 93% | C | S | S | S | CA_C2434 | Membrane associate | Transcription | Y | N | Y | Y |
| | | | | | | | | | histidine kinase with | translation | | | | |
| | | | | | | | | | HAMP domain | regulation | | | | |
| 1834077 | C | T | 92% | C | N | S | L | CA_C1684 | TYPA/BIPA type | Energy | Y | Y | Y | Y |
| | | | | | | | | | GTPase | metabolism | | | | |
| 3927304 | G | A | 92% | I | | | | | I | I | Y | N | Y | N |
| 786649 | — | T | 91% | C | FC | | | CA_C0680 | Predicted membrane | Membrane | N | N | N | N |
| | | | | | | | | | protein | proteins | | | | |
| 2640439 | C | T | 91% | C | N | E | K | CA_C2532 | Protein containing ChW- | Cell adhesion | Y | Y | Y | Y |
| | | | | | | | | | repeats | | | | | |
| 3601904 | A | — | 91% | C | FC | | | CA_C3415 | ABC-type | Transporters | N | N | N | N |
| | | | | | | | | | multidrug/protein/lipid | | | | | |
| | | | | | | | | | transport system, | | | | | |
| | | | | | | | | | ATPase component | | | | | |
| 838350 | A | — | 89% | C | FC | | | CA_C0723 | Transcriptional | Transcription | N | N | N | N |
| | | | | | | | | | regulator, AcrR family | translation | | | | |
| | | | | | | | | | | regulation | | | | |
| 3721023 | G | A | 89% | C | S | S | S | CA_C3523 | Hypothetical protein, | Hypothetical | Y | Y | Y | Y |
| | | | | | | | | | CF-7 family | proteins | | | | |
| 803924 | G | A | 88% | C | N | A | T | CA_C0695 | Altronate | Carbohydrate | Y | Y | Y | Y |
| | | | | | | | | | oxidoreductase | metabolism | | | | |
| 3478420 | C | T | 87% | C | N | G | E | CA_C3309 | Predicted membrane | Membrane | N | Y | N | Y |
| | | | | | | | | | protein | proteins | | | | |
| 3853836 | T | C | 87% | C | N | N | D | CA_C3652 | Acetolactate synthase | Amino acid | Y | Y | Y | Y |
| | | | | | | | | | | metabolism | | | | |
| 244464 | C | T | 86% | C | N | S | L | CA_C0220 | Hypothetical protein | Hypothetical | Y | Y | Y | Y |
| | | | | | | | | | | proteins | | | | |
| 899104 | G | A | 86% | C | N | M | I | CA_C0776 | NCAIR mutase (PurE)- | Nucleic acid | Y | N | Y | N |
| | | | | | | | | | related protein | metabolism | | | | |
| 658665 | T | — | 85% | C | FC | | | CA_C0569 | SACPA operon | Transcription | N | N | N | N |
| | | | | | | | | | antiterminator (sacT) | translation | | | | |
| | | | | | | | | | | regulation | | | | |

### REFERENCES

1. González-Pajuelo M, Meynial-Salles I, Mendes F, Andrade J C, Vasconcelos I, and Soucaille P. 2005. Metabolic engineering of Clostridium acetobutylicum for the industrial production of 1,3-propanediol from glycerol. Metabolic Engineering 7: 329-336.
2. González-Pajuelo M, Meynial-Salles I, Mendes F, Soucaille P. and Vasconcelos I. 2006. Microbial conversion of a natural producer, Clostridium butyricum VPI 3266, and an engineered strain, Clostridium acetobutylicum DG (pSPD5). Applied and Environmental Microbiology, 72: 96-101.
3. Nölling J, Breton G, Omelchenko M V, Makarova K S, Zeng Q, Gibson R, Lee H M, Dubois J, Qiu D, Hitti J, Wolf Y I, Tatusov R L, Sabathe F, Doucette-Stamm L, Soucaille P, Daly M J, Bennett G N, Koonin E V, Smith D R. 2001. Genome sequence and comparative analysis of the solvent-producing bacterium Clostridium acetobutylicum. Journal of bacteriology 183(16):4823-4838
4. Papanikolaou S, Ruiz-Sanchez P, Pariset B, Blanchard F and Fick M. 2000. High production of 1,3-propanediol from industrial glycerol by a newly isolated Clostridium butyricum strain. Journal of Biotechnology. 77: 191-208.
5. Vasconcelos I, Girbal L, Soucaille P. 1994. Regulation of carbon and electron flow in Clostridium acetobutylicum grown in chemostat culture at neutral pH on mixtures of glucose and glycerol. Journal of bacteriology. 176(5): 1443-1450.

1. A population of Clostridium acetobutylicum useful for producing 1,3-propanediol (PDO), wherein said population comprises at least one strain of a Clostridium acetobutylicum sp. comprising one or more mutations selected from the mutations identified in Table 1, wherein said mutations are present among the following gene families in the relative percentages of:

| Gene family and function | Minimum % |
|---|---|
| Transcription translation regulation | 12-15 |
| Transporters | 10-12 |
| Hypothetical proteins | 8-11 |
| Energy metabolism | 7-10 |
| Intergenic | 7-10 |
| Carbohydrate metabolism | 5-7 |
| Membrane proteins | 2-5 |
| Nucleic acid metabolism | 2-5 |
| Amino acid metabolism | 1-3 |
| Cell division | 1-3 |
| Sporulation | 1-3 |
| Cell adhesion | 0-1 |
| Cellulase | 0-1 |
| Glycerol metabolism | 0-1 |
| Lipid metabolism | 0-1 |
| Proteases/Peptidases | 0-1 |
| Cell motility | 0-1 |

2. The population of claim 1, wherein said population comprises at least one strain of Clostridium acetobutylicum selected from the group consisting of:
strain DG1 pSPD5 PD0001VE05c01 deposited at CNCM under accession number I-4378;
strain DG1 pSPD5 PD0001VE05c05 deposited at CNCM under accession number I-4379; and
strain DG1 pSPD5 PD0001VE05c07 deposited at CNCM under accession number I-4380.

3. The population of claim 1, wherein the at least one strain is further mutated with at least one of the following point mutations:
C is replaced with T at locus CA_C0175, position 198989 in the Clostridium acetobutylicum genome, coding for a predicted sugar phosphate isomerase, homolog of an eukaryotic glucokinase regulator (carbohydrate metabolism)
G is replaced with A at locus CA_C1300, position 1444099 in the Clostridium acetobutylicum genome, coding for an RNA polymerase sigma factor RPOD (transcription and translation regulation)
C is replaced with T at locus CA_C2670, position 2787387 in the Clostridium acetobutylicum genome, coding for a Glu-tRNAGln amidotransferase subunit A (transcription and translation regulation)
C is replaced with T at locus CA_C3339, position 3512658 in the Clostridium acetobutylicum genome, coding for an ATPase component of an ABC transporter (two ATPase domains)
C is replaced with T at locus CA_C1610, position 1752341 in the Clostridium acetobutylicum genome, coding for a branched-chain amino acid permease (transporter).

4. A method for producing 1,3-propanediol, comprising culturing a population of claim 1, in a culture medium comprising glycerine as sole source of carbon, and recovering 1,3-propanediol produced from the culture medium.

5. The method of claim 4, further comprising purifying said 1,3-propanediol.

6. The method of, claim 4, wherein the glycerine concentration in the culture medium is comprised from 90 to 120 g/L glycerine, and is optionally about 105 g/L of glycerine.

7. The method of claim 4, wherein said glycerine is provided by industrial glycerine.

8. The method of claim 7, wherein said industrial glycerine is a by-product of biodiesel production.

9. The method of claim 5, wherein said culture medium is a synthetic medium, without addition of organic nitrogen.

10. A method for producing 1.3-propanediol, comprising culturing a population of claim 2 in a culture medium comprising glycerine as sole source of carbon, and recovering 1,3-propanediol produced from the culture medium.

11. A method for producing 1,3-propanediol, comprising culturing a population of claim 3 in a culture medium comprising glycerine as sole source of carbon, and recovering 1,3-propanediol produced from the culture medium.

12. The population of claim 2, wherein the at least one strain is further mutated with at least one of the following point mutations:
C is replaced with T at locus CA_C0175, position 198989 in the Clostridium acetobutylicum genome, coding for a predicted sugar phosphate isomerase, homolog of an eukaryotic glucokinase regulator (carbohydrate metabolism)
G is replaced with A at locus CA_C1300, position 1444099 in the Clostridium acetobutylicum genome, coding for an RNA polymerase sigma factor RPOD (transcription and translation regulation)
C is replaced with T at locus CA_C2670, position 2787387 in the Clostridium acetobutylicum genome, coding for a Glu-tRNAGln amidotransferase subunit A (transcription and translation regulation)
C is replaced with T at locus CA_C3339, position 3512658 in the Clostridium acetobutylicum genome, coding for an ATPase component of an ABC transporter (two ATPase domains)
C is replaced with T at locus CA_C1610, position 1752341 in the Clostridium acetobutylicum genome, coding for a branched-chain amino acid permease (transporter).

13. The method of claim 5, wherein the glycerine concentration in the culture medium is comprised from 90 to 120 g/L glycerine, and is optionally about 105 g/L of glycerine.
